(19) United States
(12) Patent Application Publication
(10) Pub. No.: US 2011/0117022 A1
(43) Pub. Date: May 19, 2011

(54) METHOD OF USING AND ESTABLISHING AN ABSORPTION RATE LEVEL AND A NEURON FIRING LEVEL

(76) Inventor: WILLIAM J. McFAUL, Jackson, NJ (US)

(21) Appl. No.: 13/013,420

(22) Filed: Jan. 25, 2011

Related U.S. Application Data
(63) Continuation-in-part of application No. 12/897,282, filed on Oct. 4, 2010.
Application No. 12/897,282 is a continuation-in-part of application No. 12/701,076, filed on Feb. 5, 2010.
(60) Provisional application No. 61/150,470, filed on Feb. 6, 2009.

(51) Int. Cl. A61K 38/51; A61K 31/195; A61P 25/00; A61K 49/00; G06F 19/00; G06Q 50/00; G06Q 10/00

(52) U.S. Cl. 424/9.1; 514/561; 424/94.5; 702/19; 705/2

(57) ABSTRACT

A method and system used in establishing the homeostatic relationship between various amino acid neurotransmitters in the body. The method includes modulating the rate of neuron firing using GABA, glutamate or GAD.

This application is a continuation in part of U.S. patent application Ser. No. 12/897,282 filed Oct. 4, 2010, which is a continuation in part of U.S. patent application Ser. No. 12/701,076 filed Feb. 5, 2010, which in turn claims the benefit of U.S. Provisional Application Ser. No. 61/150,470, filed Feb. 6, 2009, the contents of which are incorporated herein by reference.

### BACKGROUND OF THE INVENTION

1. Field of the Invention

The present invention relates to the field of amino acid neurotransmitters. In particular, the present invention relates to the identification and regulation of substances that control the homeostatic relationship of pairs of amino acid neurotransmitters.

2. Description of the Related Technology

All forms of life are supported by a variety of substances; including but not limited to hormones, proteins, peptides, amino acids, minerals (ions), vitamers (chemicals or compounds that function as vitamins) and bacteria, minerals, genes, enzymes, lipids (i.e. fatty acids), carbohydrates, amino acids, ions and gasotransmitters. These substances create the homeostasis essential to maintain life for humans as well as plants and animals.

Current scientific findings have not identified that the amino acid neurotransmitters—glutamate and glycine, aspartate and gamma aminobutyric acid (GABA) as well as aspartate and glutamate function as pairs to regulate the body's rate of cellular absorption and the firing rate of neurons and signaling mechanisms throughout the body. In addition, medical science has failed to recognize the fact that the homeostatic relationships between these amino acid neurotransmitters are regulated by other substances that can assist in placing the amino acid neurotransmitter back in homeostatic equilibrium.

Therefore, there is a need in the field to identify and regulate the homeostatic relationships between the amino acid neurotransmitters that are important to the body's functioning.

### SUMMARY OF THE INVENTION

Another object of the invention may be determining whether or not aspartate and glutamate are within homeostatic balance.

Another object of the invention may be to determine whether or not glutamate and glycine are within homeostatic balance.

Still yet another object of the invention may administering GABA, glutamate, or GAD when aspartate and glutamate are not in homeostatic balance.

An aspect of the present invention may be A method for establishing homeostasis for neuron firing comprising: measuring a level of aspartate in an individual; measuring a level of glutamate in the individual; calculating a ratio of the aspartate level to the glutamate; and administering GABA, GAD or glutamate to the individual when the ratio is not within a predetermined range; and wherein the GABA, glutamate or GAD is administered by provision of orally administered substances, intravenously administered solutions, modification of dietary supplements, sublingually administered, injections, dermal patches or some other means.

Still yet another aspect of the present invention is may be a system for generating individual reports comprising: a database; test data obtained from measuring a level of aspartate in an individual; measuring a level of glutamate in the individual and calculating a ratio of the aspartate level to the glutamate; wherein the test data is stored on the database; a central server connected to the database; a central computer connected to the central server; wherein test data is compiled and correlated at the central computer; and an individual report generator located at the central computer.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 shows a diagram illustrating the homeostatic relationship.

FIG. 2 shows a diagram illustrating the homeostatic relationship of glutamate, glycine and cysteine.

FIG. 3 is a flow chart of the method for establishing homeostasis of glutamate, glycine.

FIG. 4 shows a diagram illustrating the homeostatic relationship of aspartate, glutamate and GABA.

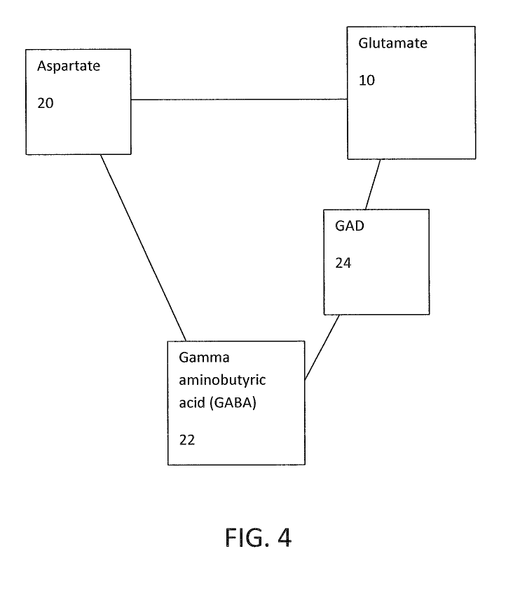

FIG. 5 is a flow chart of the method for establishing homeostasis of aspartate and glutamate.

FIG. 6 is a schematic diagram showing the system for measuring the levels of amino neurotransmitters contained within the body.

FIG. 7 is a flow chart showing the steps involved in using the system shown in FIG. 6.

### DETAILED DESCRIPTION OF THE PREFERRED EMBODIMENT(S)

All forms of life are supported by modulated pairs of substances; including but not limited to hormones, proteins, peptides, amino acids, minerals (ions), vitamers (chemicals or compounds that function as vitamins) and bacteria minerals, genes, enzymes, lipids (i.e. fatty acids), carbohydrates, amino acids, ions and gasotransmitters. These substances create the homeostasis essential to maintain life for humans as well as plants and animals.

Several terms are used herein and are defined as follows:

The term “modulator” means an agent, substance or mechanism that facilitates the maintenance of homeostasis between pairs. “Pairs” may be hormones, minerals, proteins, amino acids, bacteria, virus, and natural processes. The agent can decrease intensity of stimulatory processes or substances, or increase intensity of compensatory ones.

The term “catalyst” means the driver which initiates modulation to take place. Such catalysts can include enzymes, hormones, RNA, and countless signaling mechanisms.

The term “substance” refers to compounds, elements, ions and molecules and combinations thereof.

The term “neuron” means the signaling molecules or signaling mechanisms.

The term “Homeostatic Relationship (Homeostasis)” means the balance or equilibrium between two substances. The relationship does not have to be 50%-50% but can vary depending upon the two substances in question.

The invention addresses the diagnostic analysis of the corollary relationships between amino acids that provide and maintain homeostasis; i.e. pairs which include substances that are stimulatory/excitatory (e.g. glutamate and aspartate) and those that are compensatory/inhibitory (e.g. glycine and gamma aminobutyric acid). While medical science has identified the nature of these amino acids (e.g. excitatory or inhibitory), they have not identified their specific roles, the relationships between them or the existence of substances that modulate the relationships between them.

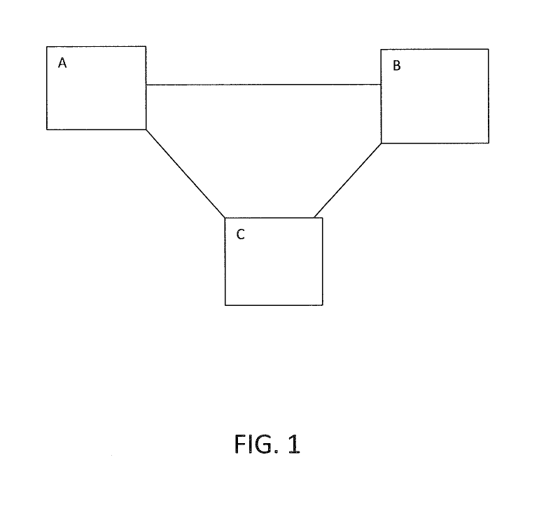

The concept of modulation in relation to homeostatic pairs is illustrated in FIG. 1. This example illustrates a relationship between two substances, A and B, and the existence of a third substance C that serves to support A or B in order to attempt to maintain an adequate level of homeostasis (balance) between the two substances.

Current methodologies do not account for processes associated with fluctuations of or imbalances between cellular absorption rates and the rates of firing of neurons. Medical science knows that minerals and certain amino acids bind to various types of cells. It is assumed that the binding process occurs as the mechanism by which all cells absorb all substances (also referred to as intake or uptake). Instead, the rate of cellular absorption is determined by the homeostatic relationship between glutamate and glycine.

The natural mechanism that facilitates the homeostatic relationship for maintaining adequate cellular absorption is the pairing of glutamate and glycine and utilization of cysteine. In instances where an imbalance occurs that has the potential of disrupting homeostasis, cysteine serves to modulate the imbalance.

A low or slow rate of cellular absorption will retard the body's ability to process substances, other than neurons, to maintain homeostatic relations or other systems and processes throughout the body. Conversely, a greater or higher rate of cellular absorption will disrupt homeostatic relationships throughout the body by accelerating systems and processes. Examples of an accelerated rate of absorption are allergic reactions resulting from substances that might not impact individuals with lower rates of cellular absorption and cancers that may result when cells absorb greater levels of toxins.

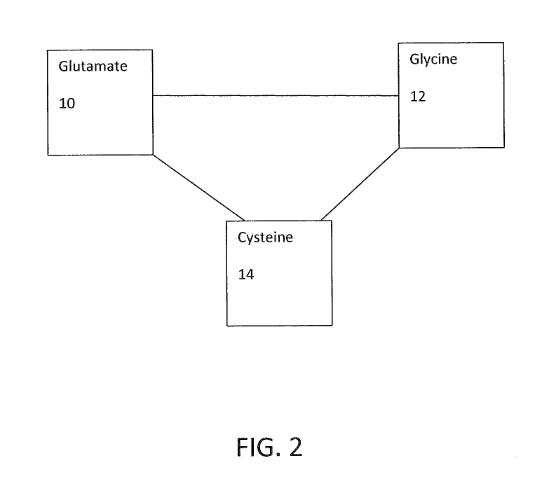

For example, homeostasis for cellular absorption rates is determined by the pairing of glutamate 10, which controls the increase in absorption rate with glycine 12, which decreases the absorption rate. The amino acid cysteine 14 serves as the modulator for the process. Application of cysteine 14 increases the level of glycine 12 within the body. This relationship is diagrammatically illustrated in FIG. 2. Additionally, it should be understood that while specific processes and roles are noted herein as being affected by the pairs, other processes and roles may also be affected.

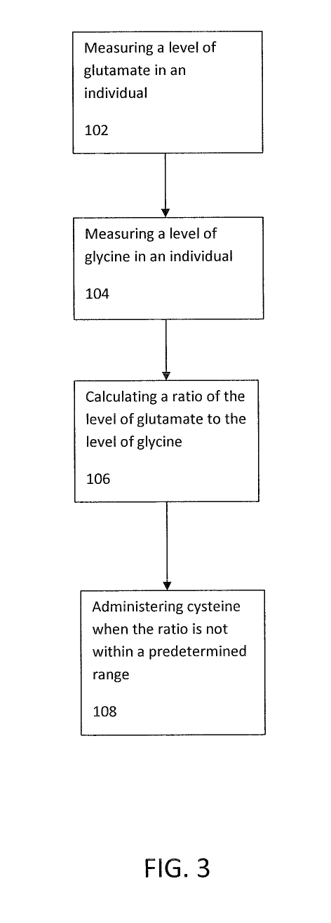

FIG. 3 shows the method of returning the levels of glutamate 10 and glycine 12 to homeostatic balance. In step 102 the level of glutamate 10 is measured in an individual. The level of glutamate 10 may be measured by a variety of diagnostic tests that include but are not limited to the analysis of diagnostic imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat; tears; saliva; mucus; phlegm; hair; fingernails; bone marrow. The measured level of glutamate 10 in the body is expressed in various units depending upon the test employed in order to measure the level of glutamate 10. This level can be expressed by the variable X. There is a preferred range in which X may fall that varies depending upon the type of test that is used in order to measure the level of glutamate 10. In an embodiment of the present invention a standard type of test is used, such as blood test.

In step 104 the level of glycine 12 in an individual is measured. This level can be measured using one of the various processes listed above. The measured level of glycine 12 is preferably expressed in the same units of measurement as that used in the measurement of glutamate 10 so as to more readily compare the levels with respect to each other. This level can be expressed by the variable Y. There is a preferred range in which Y may fall that varies depending upon the type of test that is used in order to measure the level of glycine 12. In an embodiment of the present invention a standard type of test is used, such as a blood test.

In step 106 the level of glutamate 10 that has been measured, X, is compared to the level of glycine 12, Y, in order to obtain ratio. This ratio, X/Y, can be expressed by the variable Z. The variable Z preferably falls within a predetermined range which expresses whether or not glutamate 10 and glycine 12 are within homeostatic balance. The predetermined range may be established by surveying a population of individuals and determining means ranges from surveyed population. This range may be refined over time as the surveyed population increases.

In step 108, if it is determined that the ratio Z is not within the predetermined range, then cysteine 14 is administered to the individual in order to restore the glutamate 10 and glycine 12 levels to a preferred range. Administration of cysteine 14 may be orally administered, e.g., pills, solutions, lozenges, etc.; intravenously administered solutions; modification of dietary supplements and/or other standard methods of delivery. After a period of time after administration, the levels of each of the substances may be retested in order to ascertain whether or not the administration was effective. Additionally, the level of cysteine 14 may be measured in order to determine if more should be administered.

An example of this is provided below using hypothetical numbers so as to make understanding of the process easier. Measured results may further be normalized to a set scale in order to make comparison and calculation easier provided that all values are expressed in the same units. Actual numbers from a test such as a blood test are typically expressed in units of mmol/L.

Measuring glutamate 10 via a blood test results in a number for X that results in a number 3, from within a range of 1-10. Measuring glycine 12 via a blood test results in a number for Y of 5 from within a range of 1-10. The number Z that results from this is 0.6. A preferred range for Z is between 0.8 and 1.2. By administering cysteine 14 to the individual the level of glycine 12 is increased so that on a subsequent test the level of glutamate 10 results in the number 4 for X, while the number for Y remains at 5. This results in a Z of 0.8, which falls within a predetermined range, say from 0.5 to 1.0, and indicates that glutamate 10 and glycine 12 are in homeostatic balance and no further application of modulator cysteine 14 is required.

Another example of a modulated homeostatic relationship is that certain cells emit neurons while other cells receive and convert these neurons into various activities. The rate of neuron emission is determined by a homeostatic relationship between aspartate 20 for emission and glutamate 10. The levels of aspartate 20 are inhibited (lowered) by GABA 22 (Gamma-aminobutyric acid).

Aspartate 20 governs the increase in the firing rate and GABA 22 governs the decrease in firing rate. GAD 24 (Glutamic acid decarboxylase) is the catalyst used to convert glutamate 10 into GABA 22 in order to enable GABA 22 to modulate, i.e. reduce, excessive firing rate in order to maintain a homeostatic relationship between the rate of neural firing of some cells and the ability of other cells to absorb the neurons.

If the rate of neural firing increases, levels of glutamate 10 should increase in order to create the enzyme GAD 24 to insure the firing rate of neurons is reduced to maintain homeostasis between firing and absorption rates.

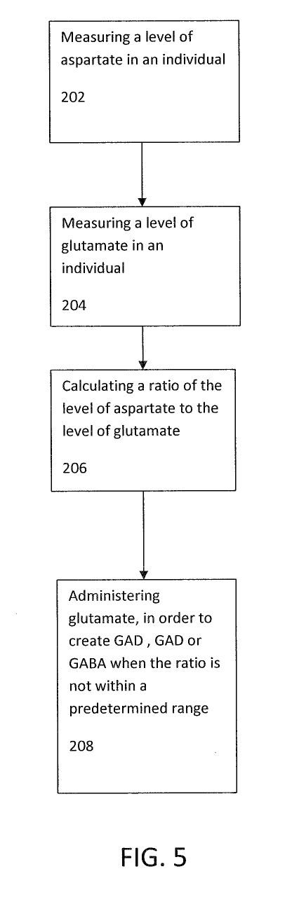

FIG. 5 shows the method of returning the levels of aspartate 20 and glutamate 10 to homeostatic balance. In step 202 the level of aspartate 20 is measured in an individual. The level of aspartate 20 may be measured by a variety of diagnostic tests that include but are not limited to the analysis of diagnostic imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat; tears; saliva; mucus; phlegm; hair; fingernails; bone marrow. The measured level of aspartate 20 in the body is expressed in various units depending upon the test employed in order to measure the level of the aspartate 20. This level can be expressed by the variable X. There is a preferred range in which X may fall that varies depending upon the type of test that is used in order to measure the level of aspartate 20. In an embodiment of the present invention a standard type of test is used, such as a blood test.

In step 204 the level of glutamate 10 in an individual is measured. This level can be measured using one of the various processes listed above. The measured level of glutamate 10 is preferably expressed in the same units of measurement as that used in the measurement of aspartate 20 so as to more readily compare the levels with respect to each other. This level can be expressed by the variable Y. There is a preferred range in which Y may fall that varies depending upon the type of test that is used in order to measure the level of glutamate 10. In an embodiment of the present invention a standard type of test is used, such as a blood test.

In step 206 the level of aspartate 20 that has been measured, X, is compared to the level of glutamate 10, Y, in order to obtain ratio. This ratio, X/Y, can be expressed by the variable Z. The variable Z preferably falls within a predetermined range, which expresses whether or not aspartate 20 and glutamate 10 are within homeostatic balance. The predetermined range may be established by surveying a population of individuals and determining means ranges from surveyed population. This range may be refined over time as the surveyed population increases.

In step 208, if it is determined that the ratio Z is not within the predetermined range, then, GABA 22, glutamate 10 that is converted to GABA 22 by GAD 24, GAD 24 or GABA 22 itself itself is administered to the individual when the ratio is not within a predetermined range. The substance is administered to the individual in order to restore the aspartate 20 and glutamate 10 levels to a preferred range. Administration may be accomplished by provision of orally administered substances e.g. pills, solutions, lozenges, etc.; intravenously administered solutions; modification of dietary supplements and/or other standard methods of delivery. After a period of time, the levels of each of the substances may be retested in order to ascertain whether or not the administration was effective. Additionally, the level of GAD 24, GABA 22 or glutamate 10 may be measured in order to determine if more should be administered.

An example of this is provided below using hypothetical numbers so as to make understanding of the process easier. Measured results may further be normalized to a set scale in order to make comparison and calculation easier provided the all values are expressed in the same units. Actual numbers from a test such as a blood test are typically expressed in units of mmol/L.

Measuring aspartate 20 via a blood test results in a number for X that results in a number 5, from within a range of 1-10. Measuring glutamate 10 via a blood test results in a number for Y of 3 from within a range of 1-10. The number Z that results from this is 1.6. A preferred range for Z is between 0.8 and 1.20. By administering glutamate 10, GABA 22 or GAD 24 to the individual the level so that on a subsequent test the level of glutamate 10 results in the number 4.5 for Y, while the number for X remains at 5. This results in a Z of 1.1, which falls within the predetermined range and indicates that aspartate 20 and glutamate 10 are in homeostatic balance and no further application of modulator GABA 22 is required.

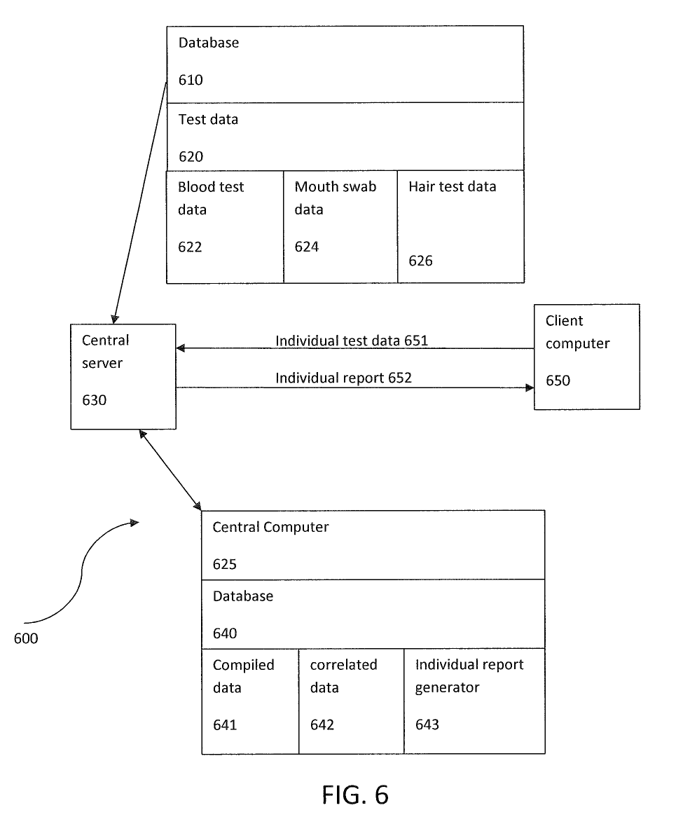

The usage of these methods can also be used for the design of devices to correlate the relationship between the various neurotransmitters contained in the body as listed in this invention. These devices can include but should not be limited to ones used within the veins and arteries, ongoing assessment as implanted devices, monitoring devices for home use or those used to perform diagnostic assessments in physicians offices, hospitals, diagnostic laboratories, home care or any other setting where the analysis of these gases contained in the blood are measured and compared to each other to perform assessments to maintain proper health and well being. A schematic of the system diagram is shown in FIG. 6.

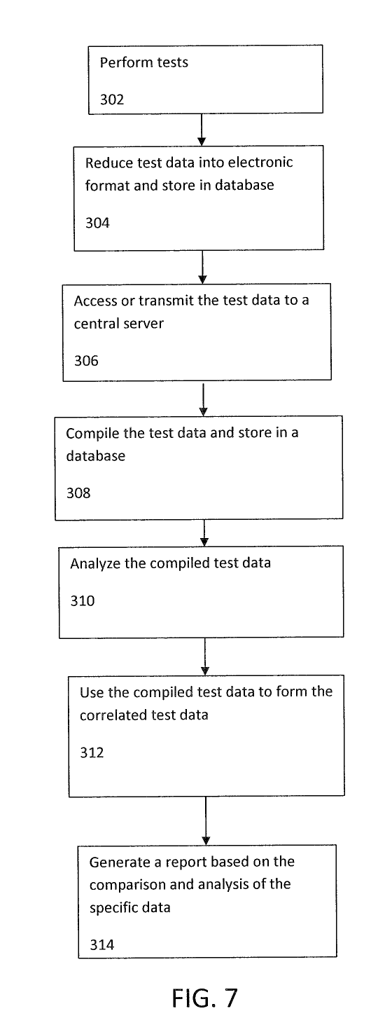

FIG. 6 shows a personalized report generator system 600 designed and arranged in accordance with an embodiment of the present invention. It should be understood that the system 600 shows the basic components of the system and that more or less individualized components may be provided. FIG. 7 is a flow chart showing the steps involved in using the system 600.

The personalized report generator system 600 comprises a database 610 in which may be stored various test results, which are stored in the database 610 as test data 620. The test data 620 is in a format that is capable of being manipulated by computers, processors, etc. The test data 620 may comprise various neurotransmitter levels. In an exemplary embodiment of the present invention and shown in step 302, tests are performed on individual patients. In step 304, the results of these tests are then reduced into an electronic format in order to produce the test data 620. For example, the database 610 may store blood test data 622, mouth swab data 624 and hair test data 626. Other tests may include urine; fecal matter; collagen; chyle; interstitial fluid (tissue fluid); lymph; extracellular fluid; amniotic fluid; sweat tears; saliva; mucus; phlegm; hair; fingernails; and bone marrow. Each of these various tests may screen for a plurality of neurotransmitters. As an example, the blood test data 622 may screen for aspartate, glutamate, GAD or GABA.

The test data 620 may be organized and stored at the database 610. Alternatively, satellite locations may each maintain their own databases 610 that control their own records. In step 306, these locations may be accessed periodically by the central server 630 or the various databases 610 may transmit their results to a central location that is then accessible by a central server 630. Preferably the test data 620 is only accessed or transmitted when a certain threshold number of results are obtained. For example the test data 620 may not be sent to the central server 630 until at least one hundred tests are prepared to be transmitted.

In step 308, the transmitted test data 620 is compiled and stored as compiled test data 641. The central server 630 may keep the sent or accessed compiled test data 741 stored at its location in the central database 640, or the system 600 may provide a distributed storage system that stores the compiled test data 641 at external locations as well as the central database 640. However it should be understood that when compiled test data 641 is stored at distributed sites the system still functions as if they were stored at the central database 640. The compiled test data 641 may be stored so that the origin of the data is maintained in an anonymous fashion. In some instances tracking of the origin of a test result may only occur when a personalized report is requested.

In step 310, the compiled test data 641 is then analyzed in order to determine significant figures for conducting analysis. The analysis may be accomplished with the usage of a computer(s) 625 of which the server 630 may be a part of, as well as the database 610. The analysis may be accomplished by statistically compiling and correlating the results for one specific test or by compiling the data for two separate items that are believed to have a relationship. Specific values from the plurality of compiled test data 641 having a similar feature may be analyzed. This may be accomplished through the application of known statistical algorithms. In step 312, the statistically compiled test data 641 may be used to form the correlated data 642. In step 314, the statistically correlated data 642 are then used by the individual report generator 643 to form the personalized medical prescription and diagnosis. Upon receipt of individual test data 651 from a client computer 650, the individual report generator 643 processes the data and transmits an individual report 652 to a client computer 650.

The compiled test data 641 may be periodically updated from the databases 610 and through the addition of more collected test data. The updating of the compiled test data 641 may provide additional information with which to refine the correlated data 642 and to refine the ranges used by the individual report generator 643 when producing the personalized medical prescription and diagnosis. Additionally the updating of the compiled test data 641 may be a continuous feature wherein the addition of new information is immediately reflected in the correlated data 642. This may provide for a constantly fluid analysis of compiled test data 641 that may reflect changes in population and diets in society.

In addition to the analysis of levels of amino acids, including amino acid neurotransmitters, various existing and yet to be developed diagnostic processes that measure outcomes involving these substances can be utilized. Examples of such methodologies include but are not limited to EEG, PET scans, use of MEG machines, SPECT analysis, functional and diffusion MRI technologies as well as other iterations thereof (fMRI and dMRI respectively), CT scans, and ultrasound.

The correlation between base values expressed when the range of homeostasis is determined and an individual's test results can be used as a means of assessing levels or disruptions between pairs or the existence of increases in the levels of modulators in order to maintain homeostasis. It is also contemplated that methods of diagnostic analysis may also be used to detect the presence of catalysts as markers for the existence of a disruption within a modulated pair that the body is in the process of remediating. Detection of such catalysts may be included as a factor in the process of analyzing the correlation of substances within modulated pairs.

The present invention provides the biological foundation that will enable the monitoring of relationships of modulated pairs of amino acid neurotransmitters as they relate to medical care; including wellness, prevention and treatment pertaining to the mind and body.

The following are examples of the benefits of assessing and monitoring the firing rate of neurons and the rate of cellular absorption.

Medications and herbal preparations can be ineffective or cause serious consequences if dosage does not correspond properly to absorption. Creating a baseline assessment of an individual's homeostatic absorption rate can provide a foundation from which dosing can be tailored to meet specific requirements.

Assessment of homeostatic imbalances of cellular absorption rates, and neural firing rates and the relationship between firing rates and absorption rates can enable therapies to correct these disruptions instead of waiting to the point where mental or physical consequences will require expensive or sophisticated treatment of ailments or diseases that are the outcomes.

Rates of activity and absorption will dictate the rate of use or dissipation of nutrients essential to provide energy to maintain bodily processes and functions; including but not limited to the creation and maintenance of the body's defenses (i.e. immune system).

Assessment of the neural firing rates in the brain and the ability to absorb these neurons as a means of determining potential root causes of neuro-developmental or neurodegenerative diseases and disorders as well as the efficacy of treatment regimens. Such diseases and disorders can include but should not be limited to Parkinson's, schizophrenia, Asperger's, attention deficit hyperactivity disorder, obsessive compulsive disorder, depression and Alzheimer's.

Assessment of the impact of various substances on the firing rate of neurons and signaling mechanisms and their impact on the body's rates of activities. This aspect of the invention can enable evaluation of the impact of disruptions from imbalances in amino acid neurotransmitters that may result in signaling mechanisms that may cause hormone, organ or gland imbalances. Assessment of these processes can prevent diseases or enable treatment regimens for diabetes, obesity resulting from disruptions for when to store fat, thyroid imbalances as well as numerous other disease entities.

Assessment of a cell's ability to absorb hormones, immune defenses and other essential substances in order to maintain homeostasis throughout the body.

Provide a biologically verifiable means to gauge the efficacy between branded drugs, genetic equivalents, therapeutic equivalents, herbal remedies and other substances and treatments to offer effective means of attaining or maintaining homeostatic levels of cellular absorption, neural firing rates and the relationship between these two processes.

Cellular absorption is needed for all substances in the body; not only neurons. The process for absorption of substances by cells can vary but the rate of those activities is determined by the relationship between glutamate, glycine and cysteine.

The rate of absorption is maintained by the homeostasis relationship between glutamate and glycine as modulated when cysteine modulates glycine. Also the rate of neuron firing is maintained by the homeostatic relationship between aspartate and glutamate as modulated by gamma aminobutyric acid (GABA) that has been converted from glutamate by GAD to create GABA.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method for establishing homeostasis for neuron firing comprising:
measuring a level of aspartate in an individual;
measuring a level of glutamate in the individual;
calculating a ratio of the aspartate level to the glutamate; and
administering GABA, GAD or glutamate to the individual when the ratio is not within a predetermined range; and
wherein the GABA, glutamate or GAD is administered by provision of orally administered substances, intravenously administered solutions, modification of dietary supplements sublingually administered, injections, dermal patches or some other means.

2. The method of claim 1, wherein GABA is administered to the individual until the individual establishes a ratio within the predetermined range.

3. The method of claim 1, wherein measuring the level of aspartate comprises measuring the level of glutamate in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

4. The method of claim 1, wherein measuring the level of glutamate comprises measuring the level of GABA in a bodily excretion selected from the group consisting of blood and its components; urine; fecal matter; collagen; chyle; interstitial fluid; lymph; extracellular fluid; amniotic fluid; sweat; tears; saliva; mucus; phlegm; hair; fingernails; bone marrow.

5. The method of claim 4, wherein measuring the level of aspartate and measuring the level of GABA comprises measuring the level of aspartate and GABA in the same bodily excretion.

6. The method of claim 5, wherein the bodily excretion is blood.

7. The method of claim 1, wherein GABA is administered to the individual until the individual establishes a ratio within the predetermined range.

8. The method of claim 1, wherein GABA is administered to the individual until homeostasis between neuron firing and cellular absorption is achieved.

9. The method of claim 1, wherein GAD is administered to the individual until the individual establishes a ratio within the predetermined range.

10. The method of claim 1, wherein GAD is administered to the individual until homeostasis between neuron firing and cellular absorption is achieved.

11. The method of claim 1, further comprising:
re-measuring the level of aspartate in the individual;
re-measuring the level of glutamate in the individual; and
determining whether or not homeostasis has been obtained.

12. The method of claim 1, wherein the GAD is administered by provision of orally administered substances, intravenously administered solutions or modification of dietary supplements, use of sublingual substances, dermal patches or injection into the body's interstitial fluid.

13. The method of claim 1, wherein GABA is administered by provision of orally administered substances, intravenously administered solutions, modification of dietary supplements, use of sublingual substances, dermal patches or injection into the body interstitial fluid.

14. A system for generating individual reports comprising:
a database;
test data obtained from measuring a level of aspartate in an individual;
measuring a level of glutamate in the individual and calculating a ratio of the aspartate level to the glutamate; wherein the test data is stored on the database;
a central server connected to the database;
a central computer connected to the central server; wherein test data is compiled and correlated at the central computer; and
an individual report generator located at the central computer.

15. The system of claim 14, further comprising an individual report, wherein the individual report is transmitted to a client computer.

16. The system of claim 15, wherein the individual report comprises a personalized medical prescription.

17. The system of claim 16, wherein the personalized medical prescription instructs administration of GABA, GAD or glutamate to the individual.

18. The system of claim 17, wherein the instruction to administer GABA, GAD or glutamate occurs when the ratio is not within a predetermined range.

19. The system of claim 14, wherein the system compiles test data.

20. The system of claim 19, wherein the compiled test data is used to form correlated test data.
